(19) United States
(12) Patent Application Publication
(10) Pub. No.: US 2004/0133265 A1
(43) Pub. Date: Jul. 8, 2004

(54) Medical device for intraluminal endovascular stenting

(76) Inventor: Niall Duffy, (US)

Correspondence Address: MEDTRONIC VASCULAR, INC., IP LEGAL DEPARTMENT, 3576 UNOCAL PLACE, SANTA ROSA, CA, 95403, US

(21) Appl. No.: 10/741,840

(22) Filed: Dec. 18, 2003

(30) Foreign Application Priority Data
Sep. 12, 2001 (IE) S2001/0828
Aug. 29, 2002 (WO) PCT/IE02/00125

(51) Int. Cl. A61F 2/06

(52) U.S. Cl. 623/1.16; 623/1.35

(57) ABSTRACT

An improved medical device for intraluminal endovascular stenting comprises a stent having a hollow cylindrical body fabricated from a plurality of circumferentially extending rings having an undulating series of peak and valleys. Links join the adjacent rings and are shaped to promote flexibility of the stent during its delivery to a treatment site and adequate scaffolding of a vessel following its deployment at the treatment site. Improved access to side-branches off the vessel is achieved by providing enlarged cell size for the stent when expanded, whilst maintaining adequate scaffolding. This is achieved by interrupting the straight sections between adjacent peaks and valleys by an inflection point at which one arm of one link is connected to join adjacent rings together.

This application is based on PCT Application No. PCT IE02/00125, filed 29 Aug. 2002, which claims priority of IE Short Term Application No. S2001/0828 filed 12 Sep. 2001, now pending.

### BACKGROUND OF THE INVENTION

This invention relates to intraluminal endovascular stenting, a method by which a prosthesis is inserted into a body lumen and expanded so as to reopen a wholly or partially blocked vessel wall and prevent the vessel from recollapsing into the lumen. Endovascular stenting is particularly useful for arteries which are blocked or narrowed and is an alternative to surgical procedures that intend to bypass the occlusion.

Percutaneous transluminal coronary angioplasty (PTCA) is used to open coronary arteries which have been occluded by a build-up of cholesterol fats or atherosclerotic plaque. Typically a guidewire is steered through the vascular system to the site of therapy. A guiding catheter, for example, can then be advanced over the guidewire and a balloon catheter advanced within the guiding catheter over the guidewire. The balloon at the distal end of the catheter is inflated causing the site of the stenosis to widen. The dilatation of the occlusion, however, can form flaps, fissures and dissections which threaten re-closure of the dilated vessel or even perforations in the vessel wall. Implantation of a metal stent can provide support for such flaps and dissections and thereby prevent reclosure of the vessel or provide a patch repair for a perforated vessel wall until corrective surgery can be performed. Reducing the possibility of restenosis after angioplasty reduces the likelihood that a secondary angioplasty procedure or a surgical bypass operation will be necessary.

An implanted prosthesis such as a stent can preclude additional procedures and maintain vascular patency by mechanically supporting dilated vessels to prevent vessel collapse. Stents can also be used to repair aneurysms, to support artificial vessels as liners of vessels or to repair dissections. Stents are suited to the treatment of any body lumen, including the vas deferens, ducts of the gallbladder, prostate gland, trachea, bronchus and liver. The body lumens range in size from 1.5 mm in the coronary vessels to 30 mm in the aortic vessel.

A stent typically is a cylindrically shaped device formed from wire(s) or a tube and intended to act as a permanent prosthesis. A stent is deployed in a body lumen from a radially compressed or crimped configuration into a radially expanded configuration which allows it to contact and support a body lumen. The stent can be made to be radially self-expanding or expandable by the use of an expansion device. The self expanding stent is made from a resilient springy material while the device expandable stent is made from a material which is plastically deformable. A plastically deformable stent can be implanted during an angioplasty procedure by using a balloon catheter bearing a stent which has been crimped onto the balloon. Stents radially expand as the balloon is inflated, forcing the stent into contact with the body lumen thereby forming a supporting relationship with the vessel walls. Deployment is effected after the stent has been introduced percutaneously, transported transluminally and positioned at a desired location by means of the balloon catheter.

A balloon of appropriate size and pressure may first be used to open the lesion. A stent crimped on a balloon is advanced to the lesion site. The stent is deployed when the balloon is inflated. The stent remains as a permanent scaffold after the balloon is withdrawn. A balloon capable of withstanding relatively high inflation pressures may be preferable for stent deployment because the stent must be forced against the artery's interior wall so that it will fully expand thereby precluding the ends of the stent from hanging down into the channel encouraging the formation of thrombus.

Previous structures used as stents or intraluminal vascular grafts have included coiled stainless steel springs; helical wound spring coil made from shape memory alloy; expanding metal stents formed in a zig-zag pattern; diamond shaped, rectangular shaped, sinusoidal and other mesh and non-mesh designs. Exemplary stent devices are disclosed in U.S. Pat. No. 5,776,161 issued to Globerman, U.S. Pat. No. 5,449,373 issued to Pinchasik et al, U.S. Pat. No. 5,643,312 issued to Fischell et al, U.S. Pat. No. 5,421,955 issued to Lau et al, and U.S. Pat. No. 5,292,331 issued to Boneau.

Problems to be overcome in stent design include inadequate radial force to maintain expansion; inadequate scaffolding of tissue to the wall; pre-dilated longitudinal rigidity which negatively impacts on stent delivery; and shortening of the stent as a consequence of radial expansion. Predilation stent longitudinal rigidity is a significant shortcoming, and prevents the threading of the stent through long tortuous vessels and lesions. Shortening of the stent is also a problem, as it is important that the stent cover the entire lesion to minimize the risk of post-operative complications. Many of these problems are the result of difficult design problems resulting from the often conflicting goals of stent design. For example, it is desirable to have a high degree of scaffolding in the stent when the stent is expanded to its rated radial size so that the vessel wall will have uniform support. However, it is also desirable to have a small, relatively smooth delivered profile when the stent is mounted on the catheter to permit the stent and catheter to traverse small diameter lesions. The person skilled in the art will appreciate that as a stent with a very small delivered profile expands radially its structural elements become farther apart and create openings which reduce the amount of scaffolding available to support the vessel. A similar situation exists with respect to the conflicting goals of improved scaffolding and flexibility during catheter delivery since proper scaffolding will not be accomplished if there are few supporting structural elements and yet a stent with too many structural elements may be difficult to crimp onto the balloon catheter such that the structural elements will not abut or interfere with each other during delivery through tortuous vessels. Also, in some stents, during plastic deformation of the stent (i.e. balloon expansion) the strain is concentrated at small zones. This limits the properties of the material that can be used as well as the radial force and the expansion rate.

U.S. Pat. No. 5,776,161 issued to Globerman addresses a number of these issues. Globerman discloses an expandable stent having a small initial diameter, flexibility along its longitudinal axis prior to expansion and minimization of rigid local strain on the stent material by the presence of rotation joints which have minimal strain during stent expansion. The stent is substantially the same length before and after expansion and being flexible longitudinally when constrained, it is easy to deliver. However additional improvements in longitudinal flexibility in the crimped stent during delivery and scaffolding after delivery are still desired.

WO 00/62710 also addresses a number of the same issues. Described there is a stent having a hollow, cylindrical body made with a plurality of rings. The rings each extend circumferentially around the cylindrical body and include an undulating series of peaks and valleys. The rings are joined together by a series of links which are shaped and arranged to promote longitudinal flexibility as the stent is delivered on the catheter and effective scaffolding after deployment. The rings are provided with inflection points on some portions of the rings which extend in a generally circumferential direction for a short distance. A link is joined at one end at the inflection point on one ring and also joined at a second end at a second inflection point on an adjacent ring. This construction allows the crimped stent to flex longitudinally when it is subjected to bending forces such as those encountered during delivery of the stent and catheter through a tortuous coronary artery.

The present invention seeks to provide an improved stent of a type generally described in WO 00/62710.

### SUMMARY OF THE INVENTION

Accordingly, the present invention provides a medical stent comprising a hollow cylindrical body having a plurality of undulating rings extending circumferentially around the cylindrical body and a plurality of links connecting adjacent rings, each link having a first end joined to a first ring, a second end joined to an adjacent second ring and a substantially V-shaped intermediate curved section disposed between adjacent rings, the first and second ends being arranged to nest within the undulations of the first and second rings respectively and the curved section being arranged to nest with the curved sections of adjacent rings when the stent is in a crimped state, the undulations of the rings comprising a series of peaks and valleys and substantially straight segment joining adjacent peaks and valleys, each alternate straight section between a peak and valley being interrupted by an inflection point at which an end of one link is connected to join adjacent rings together. Thus the number of links connecting adjacent rings is the same as the number of undulations in the ring. In one preferred arrangement, each ring comprises seven repeats of peak and valley and adjacent rings are connected by seven links. This arrangement is particularly advantageous in a stent having a diameter of 4.0 mm. Other like arrangements, for example, 5, 6 or 8 links will be particularly suitable for other stent diameter sizes. According to the arrangement described, the number of connecting links is reduced thereby providing larger cell-openings on expansion of the stent from the crimped condition. The larger cell size provides improved access to side-branches off vessels in which the stent is deployed while still retaining appropriate scaffolding effect.

In a particularly preferred arrangement, adjacent rings are aligned so that they appear as mirror images of each other. This enables the link connecting these two rings to be joined to the rings at substantially the same circumferential position on the stent, giving rise to improved flexibility of the stent in the longitudinal direction. Moreover in this arrangement, the links are enabled to be configured so that circumferentially neighbouring links are precisely aligned with one another with their curved section clear of the rings. Such an arrangement allows the links to nest closely and neatly when the stent is crimped onto a balloon and to de-nest readily on expansion of the stent.

According to another aspect of the invention, there is provided a medical stent of the type described above comprising a hollow cylindrical body formed of a plurality of circumferentially extending, undulating rings and a plurality of links connecting adjacent rings, one ring defining a distal or leading edge and another ring defining a proximal or trailing edge, in which at least the leading edge is formed with a tapered or partially tapered portion extending between the edge and the circumferentially outwardly facing surface of the body. In a preferred arrangement, this edge is formed as a truncated wedge shape portion.

The invention also provides an assembly comprising a medical stent of the types described above having a tapered leading edge mounted on the balloon of a balloon catheter, in which the links are of a substantially V-shape and each link extends with the crown of the V-shape pointing circumferentially in the same direction as the folds of the uninflated balloon.

The present invention relates to a stent having a hollow, cylindrical body made with a plurality of rings which extend circumferentially around the cylindrical body and include an undulating series of peaks and valleys. Typically, the undulating peaks and valleys of the rings are formed by opposing curved segments joined to each other by substantially straight segments. The rings are joined together by a series of links which are shaped and arranged to promote longitudinal flexibility as the stent is delivered on the catheter and effective scaffolding after deployment and to prevent shortening of the stent as the stent is expanded. In particular, the links are of substantially V-shape with a first longitudinally extending arm connected to a first ring and a second longitudinally extending arm connected to a second ring.

The rings are provided with inflection points on some portions of the rings which extend between an adjacent peak and valley of the ring. At each inflection point, a portion of the ring extends in a generally circumferential direction for a short distance. Typically, the inflection point is substantially centered between a peak and a valley of the ring. A link is joined at one end at the inflection point on one ring and also joined at a second end at a second inflection point on an adjacent ring. This link joins the rings together. Preferably, the link includes at least two curved segments in the unexpanded device which are capable of deflecting to promote the tendency of the stent to flex longitudinally when it is subjected to bending forces such as those encountered during delivery of the stent and catheter through a tortuous body vessel. Also preferably, the short portion of the ring at the inflection point which extends generally circumferentially has a length measured circumferentially which is at least as great as the width of the link to which it is attached. Preferably, the circumferential length is no more than about twice the width of the link to which it is attached. This promotes the scaffolding provided to the vessel by the expanded stent since the links can be fit together closely in a nested arrangement with the undulations of the rings as the stent is crimped on the balloon catheter. By “nest”, “nested” or nesting” herein we mean that the elements are conformally arranged such they can be in very close proximity when the stent is crimped onto the catheter but without substantial contact that would affect the ability of the various elements to move in relation to each other as the stent and catheter are advanced through a tortuous body vessel or as the stent is deployed at the site of use by expanding it radially.

Alternate straight segments between the peaks and valleys of the ring are interrupted by an inflection point which produces a offset portion in the straight segment in a generally circumferential direction. The links can be arranged to provide flexibility whether the peaks and valleys of the rings are arranged to make the rings appear to be mirror images to each other (i.e. peaks line up with or closely approach each other) or whether the peaks and valleys are paired with each other in an in-phase relationship or any alignment of the rings intermediate to those positions. Preferably, the rings are joined by multiple links (most preferably 3 or more). The curves or bends of the connecting links are of a complimentary shape and alignment to each other such that they will nest together when the stent is crimped onto the catheter.

The preferred arrangement of the stent includes the conformal nesting of ring and link components such that the stent can be readily crimped onto a balloon or other expansion device on the catheter. The stent may be made from a tube which is cut with lasers or other techniques which are well known to those skilled in the art. The initial pattern cut into the tube includes link and ring components which cooperate with each other but which provide sufficient spacing between components that the stent can be crimped onto a catheter without causing general abutment of the ring and link components with each other and also permit longitudinal movement of the link components without disturbing the crimp of the ring components on the catheter during deployment of the stent through tortuous body vessels. The need for spacing between the components in the crimped condition and in the expanded condition must be balanced with the need to provide appropriate scaffolding of the vessel being treated whilst avoiding where possible the occlusion of side branch vessel openings.

A relatively abundant number of links provides increased scaffolding of the vessel but potentially interferes both with the ability to crimp the stent onto the catheter and with access to side branch vessels which may be or become occluded, requiring the deployment in them of a further stent. In the present invention, the inflection points can provide the spacing needed for the nesting of the ring and link components by extending the ring in a generally circumferential direction for a distance which is sufficient to accommodate the width of the link component and provide space needed between the link components and the ring components to facilitate the crimping of the stent onto the catheter. Each inflection point includes an attachment to one connecting link and links connected to circumferentially adjacent inflection points extend in opposing directions. The circumferential offset at the inflection point provides for nesting of the connecting link with the ring component on the opposite side. At the same time, the number of inflection points and links is reduced to provide improved side access clearance on expansion of the stent in the body vessel. Further improvements in side branch access can be obtained by reducing the width and amplitude of the connecting links and by broadening the crown of the V-shaped link.

The stent is arranged to be crimped on the catheter such that the stent can flex near the inflection points without significant radial expansion as the stent is subjected to bending along a longitudinal axis as it is advanced through bends in a body vessel. As a stent is advanced through tortuosities of a vessel, it is subjected to bending forces which can produce longitudinal stresses on the connector links. If the movement of connector links pulls the undulations open from their crimped position, the stent can become radially enlarged and have difficulty in crossing a narrow lesion. The potential for this problem is reduced by aligning the connection of the links with the rings at a short, circumferentially extending portion and by providing curvature in the links which are then able to flex and thereby reduce stress on the junctions between the rings and the links.

Preferably the stent configuration is such that the undulating peaks and valleys of the rings are oriented such that the rings have peaks and valleys which are paired with each other in an out-of-phase relationship, that is to say, with longitudinally adjacent rings appearing as mirror images of one another. In such a configuration, a link can be provided which interconnects with the rings at points on the rings which are substantially centered between the respective peaks and valleys of the rings and yet allows the link to nest partially between adjacent rings and partially between adjacent peaks and valleys of the rings to which it is connected.

The invention will now be described in more detail by reference to the accompanying drawings which show embodiments of a medical stent and an assembly according to the invention.

### BRIEF DESCRIPTION OF THE DRAWINGS

FIG. 1 is a flattened plan view showing a prior art stent made according to WO 00/62710;

FIG. 2 is a flattened plan view showing a stent according to the invention;

FIG. 3 is a cross-sectional view of the ringed portion of FIG. 2;

FIGS. 4a to e are views of a stent according to the invention assembled onto the balloon of a balloon catheter; in which

FIG. 4a is an elevation view from one side;

FIG. 4b is an elevation view from the opposite side;

FIG. 4c is an end view;

FIG. 4d is a perspective view; and

FIG. 4e is an enlarged detail view of the proximal portion of the stent of FIG. 4d.

### DESCRIPTION OF THE PREFERRED EMBODIMENTS

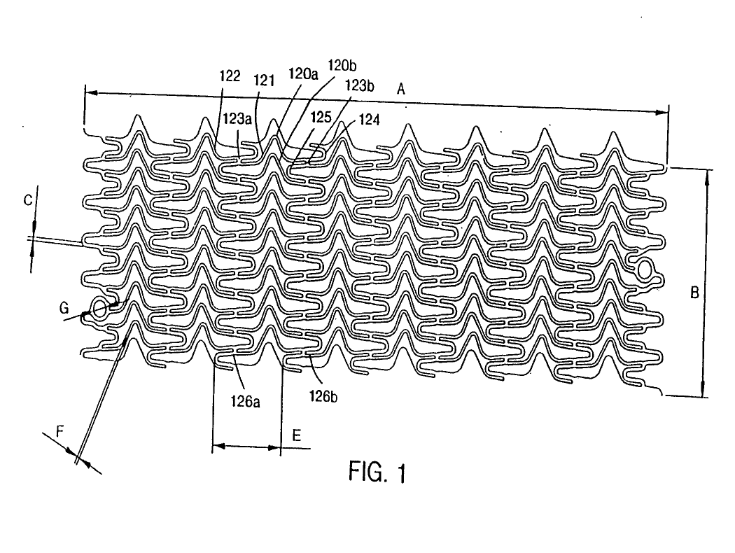

Referring firstly to FIG. 1, a prior art stent described in commonly owned International Patent Application WO 00/62710 is shown. Adjacent rings 126a-b are joined by generally inverted V-shaped links 121, 122. The stent 3 is shaped so that the adjacent peak crowns 120a-b of each link 121 will conform and nest neatly when the stent 3 is crimped onto a balloon catheter (not shown). It will be noted that every peak crown 120a-b is slightly offset from its nearest neighbor to compensate for the slight alteration in the shape of the link 121 that takes place as the stent 3 is crimped. This provides a more precise nesting of the links 121 in the crimped stent 3. Also to be noted is the relative positions of the connecting links 121, 122 with respect to an inflection point 123a. Each inflection point 123a has two links connected to it. The first connecting link 121 is connected to the inflection point 123a at a lower portion of the inflection point 123a while the second connecting link 122 is connected at an opposite side of the inflection point 123a at an upper portion of the inflection point 123a. The peaks 124 and valleys 125 of adjacent rings 126a-b are in an in-phase arrangement and the connecting links 121, 122 are connected to adjacent rings 126a-b such that a link 121 connects at one end to one ring 126a at a lower portion of the inflection point 123a and at the other end at an upper portion of the inflection point 123b. Thus, each portion of ring extending between adjacent peaks (see area A) has four links 121 connected to it, two attached to each of the two inflection points. Also to be noted in the pattern of FIG. 1 is that each peak 124 and each valley 125 has two connecting links 121, 122 extending laterally past them to join with another ring 126a-b. The connecting links 121, 122 proceed from one end attachment at an inflection point 123a-b such that they parallel the portion of the ring 126a-b and are positioned such that when the stent 3 is expanded they will extend outward from the inflection point 123a-b and assist in the scaffolding provided by the central portion of the ring 126a-b. The connecting links 121, 122 also extend past the peak 124 or valley 125 components to extend the scaffolding provided by the peak 124 and valley 125 components of the rings 126a-b toward the next ring. In particular, the connecting links 121, 122 extend upwardly past the peak 124 and valley 125 portions of the rings 126a-b into peaked portions 120a-b. This arrangement has excellent delivery characteristics and provides highly effective scaffolding for the stent 3 when it is expanded against a body lumen of the patient, yet has the disadvantage that for certain uses, the scaffolding may be excessive.

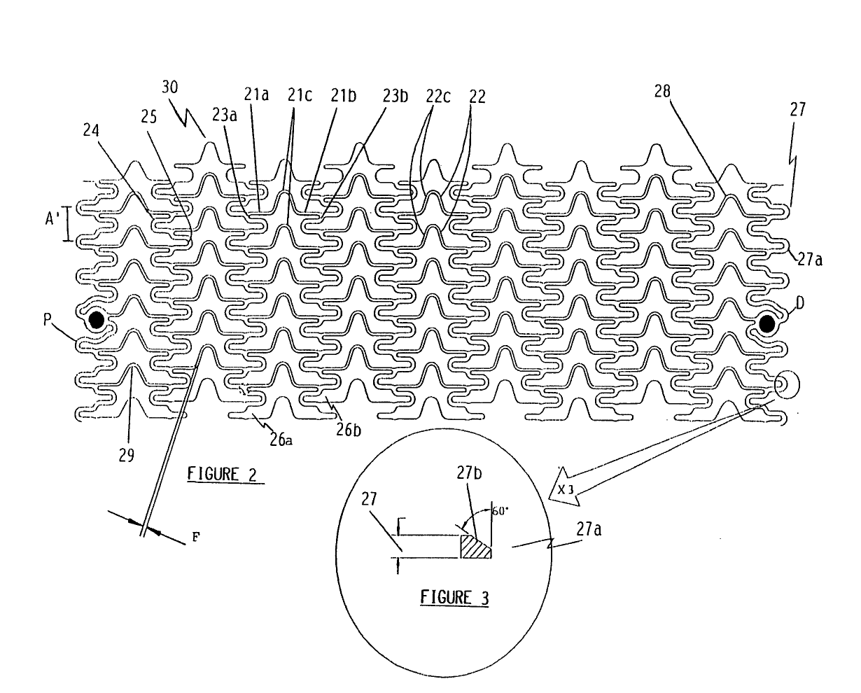

FIG. 2 shows an improved stent according to the invention. Stent 30 includes a plurality of circumferentially extending rings 26a, 26b which are linked longitudinally by a plurality of connecting links 21,22. Links 21,22 are substantially inverted V-shaped elements and are arranged in such fashion that all the links 21 which connect a pair of adjacent rings 26a,26b are substantially in register so that circumferentially adjacent links 21 nest when the stent 30 is in a crimped state and de-nest without interfering with one another or the rings 26a,26b when the stent is expanded on deployment in a body vessel. Inflection points 23a,23b are provided where the links 21 or 22 are joined to the rings. By contrast to the stent of FIG. 1, stent 30 has substantially fewer links. This is the case since each inflection points 23a, 23b has only one link 21 or 22 connected to it. Thus, each repeat in the rings 26a,26b of stent 3 (see the peak to peak distance A′) has two inflection points 23a or 23b with a total of only two links attached to each, the two links extending away from the neighbouring inflection points of a given ring in opposed longitudinal directions. By removing substantially half of the links, there are retained the benefits of the flexibility and other advantages of the stent of FIG. 1 whilst reducing the scaffolding elements contributed by those “missing” links on deployment of the stent. In particular, this feature provides the advantage of retaining a good scaffolding network whilst reducing the chances that a link member may partly or wholly obstruct the opening of a vessel which branches from the vessel in which the stent is deployed. Such side branch vessels may themselves be or become diseased or damaged in a way to make it desirable to deploy a stent or other medical device in them, but this may only be possible where a pre-placed stent does not prevent or restrict access to the side branch. Thus it is very advantageous to employ a stent which offers all the advantages of a stent of the type shown in FIG. 1 and which has the additional advantage of providing a more open-cell structure to give improved side branch access. Typically, the stent of FIG. 1 provides side branch access of in the order of 2 mm on deployment. Under like deployment conditions, the stent of FIG. 2 can provide side branch clearance access of in the order of 3 mm.

Another manner in which the improved stent 30 of FIG. 2 comprises an improvement which contributes to better side branch access is that each ring 26a,26b has 7 peaks 24 and 7 valleys 25 making 7 undulations. By contrast, the stent of FIG. 1 has 6 peaks 124 and 6 valleys 127 making 6 undulations. Both stents have an opened-out dimension which is substantially equal. Increasing the number of undulations in the ring compensates for the loss of approximately 50% of the links whilst retaining good scaffolding properties. In order to accommodate the extra undulations in the stent of the present invention, the peak to peak distance A′ in the ring has been reduced.

By comparison to the stent of FIG. 1, that of FIG. 2 has a width dimension F of the links 21,22 reduced from 0.07 mm to 0.06 mm. This reduction in link thickness aids overall flexibility of the stent. The thinner links contribute to the improved crimp profile of the stent and nesting of the links.

It has been found that the stent 30 of FIG. 2 undergoes substantially no shortening on deployment. The relative positions of the inflection points 23a, 23b remain relatively constant throughout stent expansion contributing to the avoidance of shortening.

The arrangement described in the paragraph above also has the effect that the links are compactly nested for delivery and freely denestable for deployment of the stent. By way of better explanation, it will be seen that each arm 21a,21b of link 21 will fit neatly in the space below the respective inflection point immediately above it giving a compact arrangement on crimping the stent and a free, unimpeded configuration for stent expansion.

Furthermore, it can be seen in FIG. 2 that the crowns 21c, 22c, of circumferentially adjacent V-links are in register with one another, facilitating clean nesting and denesting. Yet further it may be noted that the V-shaped portions of the links 21,22 are more flared and the crowns 21c, 22c more rounded than the sharper V-links of the stent of FIG. 1. This rounding of the V-links and their relatively thinner configuration contributes to better flexibility of the stent, better crimping down and improved side-branch access when the stent is expanded.

Yet another difference between the stent of FIG. 1 and that of FIG. 2 is that the flare of the V-shaped portion or crown of the links 28 at the distal or leading end D and the links 29 at the proximal or trailing end P of the stent is broader than that of the other links of the sent. This has the effect of strengthening the ends of the stent so as to reduce the tendency of the sent to flare radially outwardly at the ends on inflation of the balloon, as the areas of the balloon which overlap the two ends of the stent, being less restricted than the balloon section within the stent, inflate first tending to longitudinally compress or shorten the stent. This effect is avoided by strengthening the ends of the stent resulting in a more even expansion of the stent.

Yet still a further feature of the present invention which distinguishes it from the stent of FIG. 1 shall be described now with reference to FIG. 3, which is a detail cross-section view of a crown 27a of the ring 27 at the distal or leading edge of the stent 30. As can be seen in the drawing, a circumferentially outwardly facing portion at the tip of the crown 27a is formed as a chamfered surface 27b so that the outer facing parts of the stent at the leading edge have a truncated wedge shape or tapered shape. In the embodiment shown, the chamfered surface 27b describes an angle of 60°, but it will be appreciated that a wide range of angular inclinations may be employed to gain the desired effect, namely to assist the tracking of the stent through body vessels to the point of use. The tapered outer surface reduces the risks that the stent will snag as it passes through the body vessels, particularly when it must navigate tortuous paths through body lumens. Furthermore, the wedge shaped outer edge facilitates the stent in clearing occlusions encountered on the way to or at the deployment site of the stent.

The crowns at the proximal or trailing edge of the stent may or may not be chamfered in the fashion described above. Chamfering both edges offers the advantage that the stent can be assembled onto the balloon without need to consider orientation of the stent to ensure that the chamfered end is at the distal side.

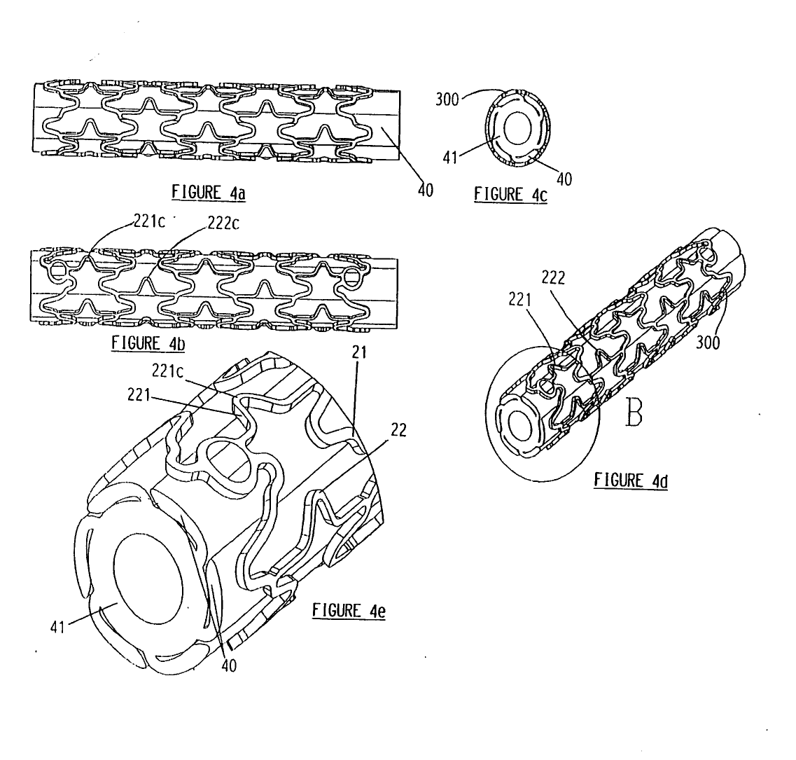

As will now be described with reference to FIGS. 4a to 4e, an advantage may be obtained from chamfering only the distal crowns of the stent. As visible in the drawing, the balloon is advantageously arranged for better compactness when deflated in a fashion wherein the longitudinal folds 40 of the balloon 41 are all neatly arranged facing in the same circumferential direction. To facilitate a smooth deployment of the stent 300 and to ensure that the links 221, 222 of the stent do not snag in the balloon folds 40 as the balloon is inflated, it is advantageous to mount the stent on the balloon with the crowns 222c of the links facing in the same circumferential orientation as the balloon folds 40. By chamfering only the distal end of the stent, an operator is facilitated in assembling the stent 30 onto the balloon in the correct fashion, since the balloon folds 40 will always be arranged in the desired orientation by the balloon manufacturing process. Therefore, the assembly person need only determine which is the chamfered edge of the stent and having done so, knows which way to turn the stent in order to assemble it onto the balloon in the preferred fashion with the links and folds in the same direction as shown in FIGS. 4a to 4e.

It will of course be understood that the invention is not limited to the specific details described herein, which are given by way of example only, and that various modifications and alterations are possible within the scope of the invention.

1. A medical stent comprising a hollow cylindrical body having a plurality of undulating rings extending circumferentially around the cylindrical body and a plurality of links connecting adjacent rings, each link having a first arm joined to a first ring, a second arm joined to an adjacent second ring and a substantially V-shaped intermediate curved section disposed between adjacent rings, the first and second arms being arranged to nest within the undulations of the first and second rings respectively and the curved section being arranged to nest with the curved sections of adjacent links when the stent is in a crimped state, the undulations of the rings comprising a series of peaks and valleys with substantially straight segments joining adjacent peaks and valleys, each straight segment between a peak and valley being interrupted by an inflection point at which an arm of one link is connected to join adjacent rings together.

2. A stent as in claim 1 wherein each ring comprises 5, 6, 7 or 8 repeats of peak and valley and adjacent rings are connected by an equal number of links.

3. A stent as in claim 1 in which adjacent rings are aligned so that they appear as mirror images of each other and the first and second arms of a link connecting these two rings are joined to the rings at substantially the same circumferential position on the stent.

4. A stent of claim 1 in which the links are configured so that circumferentially neighbouring links are precisely aligned with one another with the curved sections thereof being disposed between and clear of the rings whereby the curved sections nest neatly when the stent is crimped onto a balloon for delivery to a treatment site and de-nest readily on expansion of the stent radially at the treatment site.

5. A stent of claim 1 in which each inflection point is substantially centered between a peak and a valley of the ring.

6. A stent of claim 1 in which the inflection point includes a short generally circumferentially extending portion of a length which is at least as great as the width of the link attached to the ring at that inflection point.

7. A stent of claim 1 in which one ring defines a distal or leading edge of the stent for delivery of the stent to a treatment site and another ring defines a proximal or trailing edge of the stent, in which at least the leading edge is formed with a blunt shape to avoid the stent snagging on a vessel wall as it is delivered to the treatment site.

8. A stent of claim 7, in which the blunt edge is formed as a tapered or partially tapered portion extending between the edge and the circumferentially outwardly facing surface of the body.

9. A stent of claim 8, in which the tapered portion is formed with a truncated wedge shape.

10. A stent of claim 1 in which the terminal links closest to the distal and proximal ends of the stent are strengthened compared to the interior links of the stent to reduce outward radial flaring at the stent ends during delivery of the stent.

11. A stent of claim 10, in which each link V-shaped curved section includes a crown and the crowns of the terminal links are more rounded than the crowns of the interior links.

12. An assembly comprising a medical stent according to claim 1 wherein the medical stent is mounted on the folds of the balloon of a balloon catheter so that the V-shaped sections of the links and the folds of the balloon extend in the same circumferential direction.
